(19) United States
(12) Patent Application Publication
(10) Pub. No.: US 2004/0199169 A1
(43) Pub. Date: Oct. 7, 2004

(54) Cable clamp tool for surgical applications

(76) Inventors: Kirk C. Koons, Orlando, FL (US); Walter J. Plyter, Maitland, FL (US)

Correspondence Address: BEUSSE BROWNLEE WOLTER MORA & MAIRE, P. A., 390 NORTH ORANGE AVENUE, SUITE 2500, ORLANDO, FL, 32801, US

(21) Appl. No.: 10/718,261

(22) Filed: Nov. 20, 2003

Related U.S. Application Data
(60) Provisional application No. 60/427,895, filed on Nov. 20, 2002.

(51) Int. Cl. A61B 17/58

(52) U.S. Cl. 606/103

(57) ABSTRACT

A surgical tool for tensioning a cable positioned around a split sternum, for crimping a ferrule around the cable, and for terminating the cable at the ferrule after crimping. The tool includes a tensioning actuator for gripping and tensioning the cable to a predetermined tension and a crimping actuator for crimping the ferrule onto the cable. The crimping actuator is operative to sever a free end of the cable concurrently with crimping of the ferrule. The actuator includes an indenter to retain the ferrule therein. A method of using the tool includes looping a cable comprising a cable end fitting attached to a first end of the cable around the split sternum and passing a second end of the cable through the cable end fitting, then using the tool to tension the cable, crimp a ferrule installed on the cable, and cut the cable flush with the ferrule.

### SPECIFIC DATA RELATED TO THE INVENTION

This application claims the benefit of U.S. provisional application No. 60/427,895, filed Nov. 20, 2002.

### BACKGROUND OF THE INVENTION

The present invention relates to orthopedic surgery, and more particularly, to a method and tool for closing a sternum after surgery.

Crimping ferrules onto cables has been used as a method of securing cables in a variety of applications. More recently, the techniques of using crimping members to lock cables in a desired position have been extended to medical applications. For example, small diameter stranded cables secured by crimping members having dual lumens have been used to hold a patient's sternum together after open-heart surgery. The cable is looped around the sternum, such as between the ribs, and each end of the cable is passed through the crimping member, which is then crimped to secure the cable in place. Typically, the task of tensioning, crimping, and cutting the free ends of the cable is performed using tools designed to accomplish these tasks. Tensioning of the cable to a desired clamping tension prior to crimping, however, may be problematic. For example, patients having relatively stronger bones may require a tighter tension than patients have relatively weaker or degraded bones. Another problem with using such tools in a surgical environment is that the tools must be free from contamination when used during surgery. Consequently, the tools must be easily cleaned and able to withstand autoclaving. In addition, cables are often installed in hard to access areas of the body, so the cable crimping tool needs to be sufficiently small, maneuverable, and easy to operate for crimping and cutting the cables in confined and difficult to reach areas of the body. Conventional tools may not be easily operable in such surgical applications.

### SUMMARY OF THE INVENTION

A surgical tool for tensioning a cable positioned around a split sternum, for crimping a ferrule around the cable, and for terminating the cable is described herein as including a tensioning actuator for gripping and tensioning the cable to a predetermined tension. The tool also includes a crimping actuator for crimping the ferrule onto the cable when the predetermined tension has been reached, the crimping actuator being operative to sever a free end of the cable concurrently with crimping of the ferrule. The actuator further includes an indenter to retain the ferrule within the actuator during tensioning of the cable and crimping of the ferrule.

A method of surgically clamping a split sternum together is described herein as including looping a cable comprising a cable end fitting attached to a first end of the cable around the body parts and passing a second end of the cable through the cable end fitting. The method also includes disposing a ferrule in a ferrule receiving aperture of a clamping tool and threading the second end of the cable through the ferrule disposed in the aperture so that the ferrule abuts the cable end fitting. The method further includes wrapping the second end of the cable around a tensioning wheel of the clamping tool to apply a predetermined tension to the cable and forcing an indenter of the clamping tool into the aperture against the ferrule to crimp the ferrule around the cable. In addition, the method includes forcing the ferrule past a shearing surface of the tool to cut the cable flush with the ferrule.

### DETAILED DESCRIPTION OF THE INVENTION

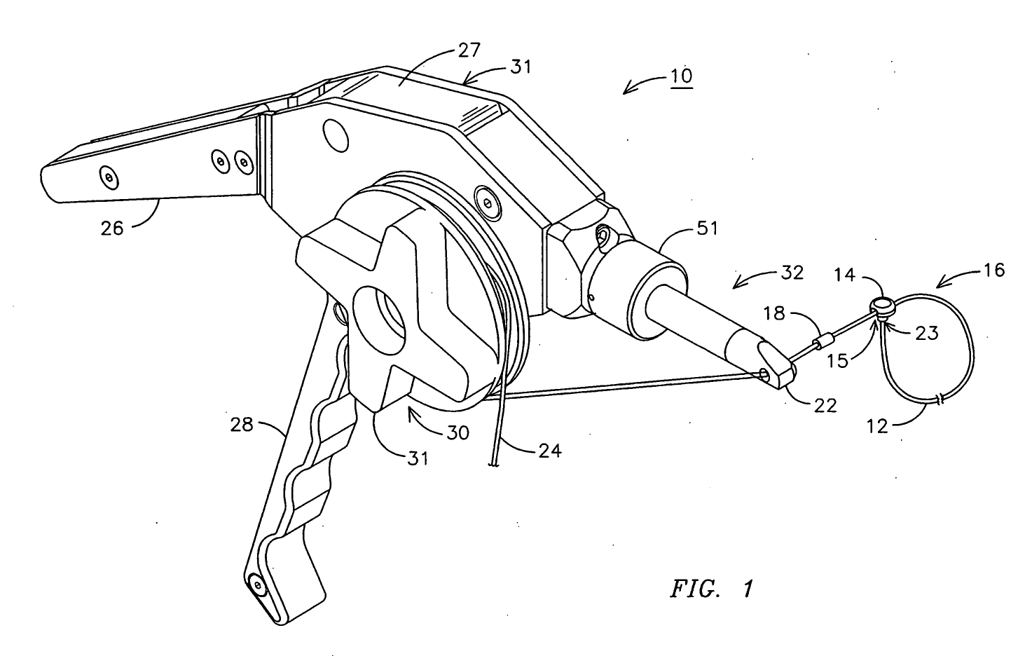

The inventors of the present invention have innovatively developed a new method and tool for securing body parts, such as clamping a split sternum together after open-heart surgery. The method will now be described with reference to FIGS. 1 and 4. FIG. 1 is a functional hardware diagram of one form of a tool for practicing the method of the present invention. The method may include providing a cable, such as the cable 12 shown in FIG. 1, having a cable end fitting 14 attached to a first end 23. The cable 12 may be made from a material suitable for use in surgical applications, such as stainless steel, and may be a monofilament or stranded type cable. The cable end fitting 23 may be shaped in the form of a smooth “button” to reduce discomfort. The cable end fitting 23 includes a passageway 15 for receiving a free end 24 of the cable therethrough. With reference to FIG. 4, the cable 12 may be installed by forming a cable loop 16 around the body parts, such as two bifurcated sections of a sternum 100, by passing the free end 24 of the cable 12 around the body parts and through the cable end fitting 14. In an aspect of the invention, the free end 24 of the cable 12 may be passed around the body parts more than one time, such as laced 102 between adjacent ribs 104, before passing the free end 24 through the cable end fitting 14. Once the cable 12 is positioned and threaded, a ferrule 18 may be threaded onto the free end 24 of the cable 12 and slid along the cable 12 to a position abutting the cable end fitting 14. Once the ferrule 18 is positioned, a desired tension may be applied to the cable 12 and maintained while the ferrule 18 is crimped onto the cable 12. For example, a relatively greater tension may be applied to the cable 12 when clamping a sternum of a patient having comparatively strong bone structure, and relatively less tension may be applied to the cable 12 when clamping a sternum of a patient having comparatively weak bone structure. After crimping the ferrule 18, the tension on the cable 12 may be maintained while cutting the cable 12 flush with the ferrule 18.

To accomplish the foregoing method for securing body parts, the inventors have developed a surgical tool 10 for tensioning a cable 12 positioned around body parts, for crimping a ferrule 18 around the cable 12, and for terminating the cable 12 while the cable 12 is tensioned to a predetermined tension. In general, the tool 10 includes a tensioning actuator, such as the cable tensioner assembly 30, for gripping and tensioning the cable 12 to a desired tension. The tool 10 also includes a crimping actuator, such as the nose 32, for crimping the ferrule 18 onto the cable 12 when the desired tension has been applied to the cable 12. The crimping actuator may be configured to sever the free end 24 of the cable 12 concurrently with crimping of the ferrule 18. In addition, the crimping actuator may be configured to retain the ferrule within the tool 10 to prevent inadvertent loss of the ferrule 18 when using the tool 10 to secure body parts. This is particularly important in a surgical environment to prevent a ferrule form being accidentally lost within a patient undergoing surgery.

As shown in FIG. 1, the tool 10 includes a nose 32 extending from a body 31 of the tool 10. The cable 12 is looped through a cable end fitting 14 forming a clamping loop 16. The ferrule 18 is seated in an aperture (not shown in this view) in the nosepiece 22 of tool 10 in a position to be crimped onto cable 12 when tension has been pulled on cable 12 by a cable tensioner assembly 30. The free end 24 of cable 12 is coupled to the cable tensioner assembly 30 to apply tension to the cable 12. Specifically, the free end 24 of the cable 12 is wrapped around the cable tensioner assembly 30 and wedged so that tension can be tangentially applied by rotating the cable tensioner assembly 30 in the same direction as the cable 12 is wrapped. For example, if the cable 12 is wrapped clockwise around the cable tensioner assembly 30, the assembly 30 is rotated clockwise to further wrap the cable 12 and apply increased tension. The wrapping of the cable 12 about the cable tensioner assembly 30 also prevents the cable from loosening while the ferrule 18 is being crimped. In one form, the cable tensioner assembly 30 provides a one-way ratcheting mechanism for maintaining tension of the cable. The ratcheting mechanism may be releasable so that the tension on the cable 12 may be released if necessary. For example, the cable tensioner assembly 30 may include a ratchet assembly 95 operative on an axle 90 of cable tensioner assembly 30. The ratchet assembly 95 may include a toothed wheel 91 and releasable pawl 93 acting on the axle 90 to provide one-way, ratcheted movement of the cable tensioner assembly 30 to allow maintaining tension on the cable 12. The pawl 93 may be manually accessible for releasing the pawl 93 from a position engaging the toothed wheel 91 to allow rotation of the cable tensioner assembly 30 in a direction opposite from a tensioning direction, for example, to allow reducing tension on a cable 12 wrapped around the cable tensioner assembly 30.

In an aspect of the invention, a tensioning wheel knob 31 of the cable tensioner assembly 30 is formed in a raised star or cross shape, providing a positive gripping surface for applying tension to the cable 12. The tool 10 further includes an upper handle 26 and a lower, movable handle 28, for example, configured in a pliers-type arrangement, for actuating the crimping of the ferrule 18 and cutting of the cable 12. The lower handle 28 may include machined finger holds for positive gripping, thereby eliminating the need for a rubberized handle which may not be usable in an autoclave. In one form of the invention, a top cover 27 of the tool 10 may be hinged to allow relatively easy access to the interior of the tool 10 for cleaning.

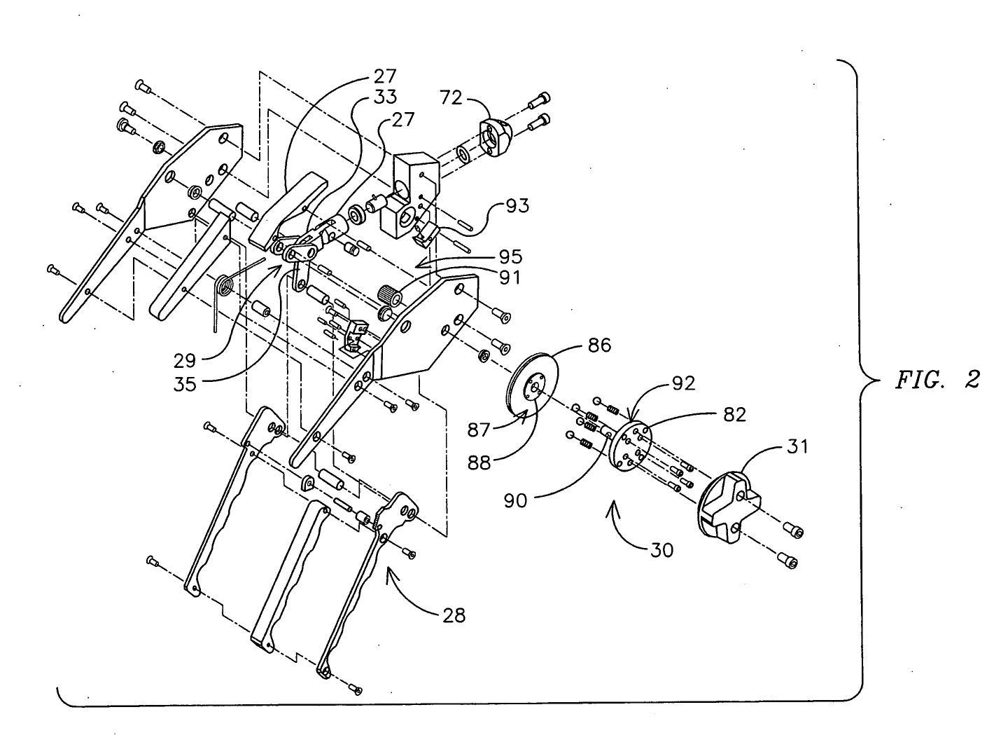

FIG. 2 is a partial exploded view of the tool of FIG. 1 including an exemplary arrangement of actuators housed within the tool 10, the movable handle 28, and the cable tensioner assembly 30. The cable tensioning assembly 30 further includes the tensioning wheel knob 31, a faceplate 86, and a tensioning wheel 82 including an axle 90. The cable tensioner assembly 30 is rotatably mounted in the tool 10 generally perpendicular to an elongate axis of the nose 32 as shown in FIG. 1. On the body side 92 of the wheel 82 where the axle 90 intersects the wheel 82, the axle 90 has a gradually flared portion (not shown) for attaching a faceplate 86 thereto. The flared portion is in the form of a truncated cone having its widest diameter at the body side 92 of the wheel 82 and its narrowest diameter corresponding to an attachment portion 88 of the faceplate 86 where the faceplate 86 is attached to the wheel 82. The attachment portion 88 of the faceplate 86 is a raised cylindrical platform having a height perpendicular to a face 87 of the faceplate 86 slightly smaller than the diameter of the safety cable 12. When the faceplate 86 is assembled to the wheel 82 at the attachment portion 88, the flared portion acts as a wedge to wedge a safety cable 12 wrapped around the flared portion of the axle 90, against the faceplate 86, thereby retaining the cable 12 so that tension can be applied by rotating the wheel 82.

The movable lever 28 is operated to actuate a piston 27 via a toggle joint 29, to provide a force parallel to the elongate axis of the nose 32 for crimping of the ferrule 18 and cutting of the cable 12. The piston 27 is attached to a driving arm 33 that is driven by a force applied to a driven arm 35 moved by the handle 28. A nose collar 72 is provided to rotatably retain the nose (not shown in this view) within the tool 10.

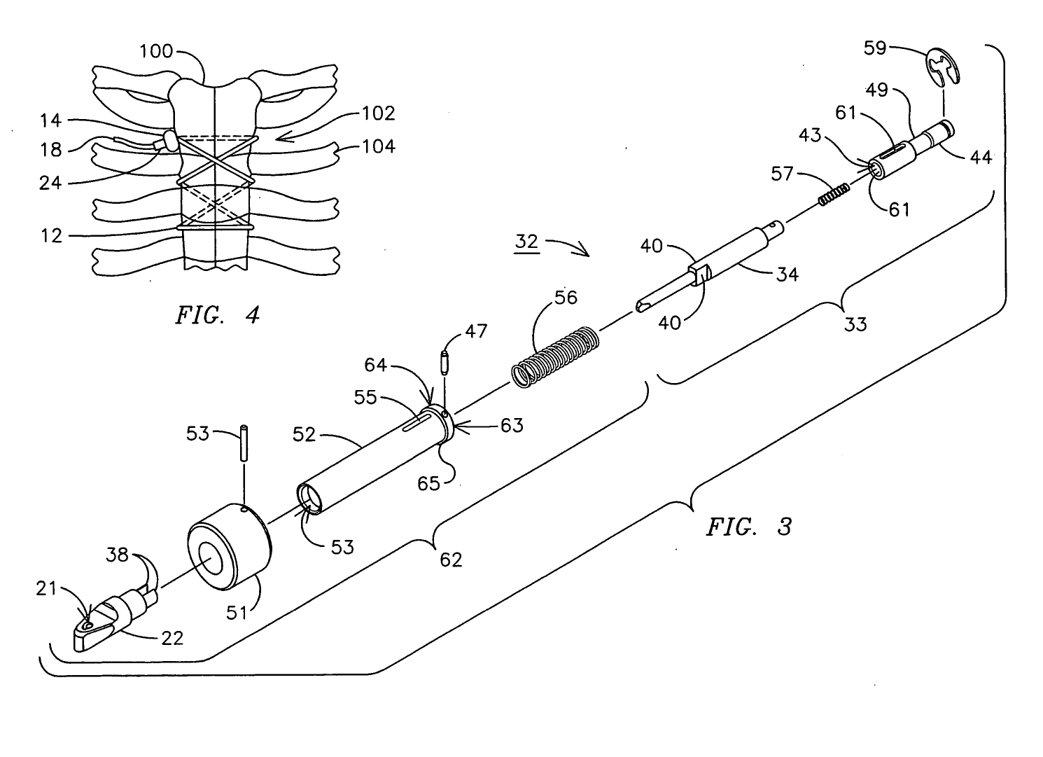

The nose 32 of the tool will now be described in further detail with respect to FIG. 3. FIG. 3 is a partial exploded view of the nose of the tool of FIG. 1. The nose 32 of the tool 10 generally includes a nosepiece 22, an indenter 34, a push rod 44, a nose extension 52, a retraction collar 51, a return spring 56, a biasing spring 57, and a retainer clip 59. The indenter 34, push rod 44, retainer clip 59, and biasing spring 57 together form a plunger assembly 33, while the nosepiece 22, nose extension 52, retraction collar 51, and return spring 56 together form a nosepiece assembly 62. It should be noted that when referring to parts comprising the nose 32, the “distal end” of a part is the end that, when assembled in the tool 10, points away from the body 31 of the tool 10. Conversely, the “proximal end” of a part is the end that, when assembled in the tool, points towards the body 31 of the tool 10. As described earlier, a ferrule 18 fits within aperture 21 in the distal end of nosepiece 22. When the plunger assembly 33 is actuated by the piston 27 in the body 31 of tool 10, the distal end of indenter 34 is pushed into the aperture 21, thereby crimping the ferrule 18 about the cable 12 passed therethrough. In an aspect of the invention, the aperture 21 is larger on an entrance side of the nosepiece 21 than it is on an exit side. The nosepiece 22 has an entrance side having a comparatively large open area, while the exit side has a comparatively small opening just suitable for passage of cable 12. The entrance side opening may have an oval configuration extending toward the distal end of nosepiece 22. Accordingly, as plunger assembly 33 is actuated, it not only crimps ferrule 18 but drives the edge of the ferrule past the exit side opening. The ferrule edge and exit side opening edge combine to act as a shear to automatically sever the free end portion 24 of the cable 12 extending out of the ferrule 18. The tool 10 thereby crimps the ferrule 18 and severs the free end 24 of the cable 12 in a single operation.

In an aspect of the invention, the proximal end of the nosepiece 22 has two alignment ears 38 configured to slidably interlock with flat portions 40 of the indenter 34 to align the plunger assembly 33 in a fixed angular orientation with respect to the aperture 21 of the nosepiece 22 for proper crimping of the ferrule 20.

The nose extension 52 includes a passageway 53, extending from the distal end to the proximal end, wherein the internal diameter of the passageway 53 is slightly larger than the outside diameter of the indenter 34 and the pushrod 44 to allow reciprocal movement of the indenter 34 and the pushrod 44 within the nose extension 52. The proximal end of the nose extension 52 includes a circumferentially enlarged cylindrical head 64 to provide a contact surface 63 for the return spring 56, and a flange 65, for rotatably seating against the nose collar 72 to prevent the nose extension 52 proximal end from being forced out of the body 31 when the plunger assembly 33 is activated. The return spring 56 is held in biased engagement against the head 64 of the nose extension 52 by the retaining clip 59 so that the spring 56 urges the plunger assembly 33 in a direction away from the aperture 21, towards the body 31 of the tool 10. The retaining clip may be a “C” clip installed in an appropriately sized circumferential groove of the pushrod 44.

The pushrod 44 may include a pushrod recess 43 in a distal portion of the pushrod 44 for receiving the retractor spring 57 and a proximal portion of the indenter 34, thereby allowing biased reciprocating motion of the indenter 34 therein. The retractor spring 57 urges the indenter 34 out of the pushrod recess 43 so that when a ferrule 18 is installed in the aperture 21, the indenter 34 is pushed into a slightly retracted position within pushrod recess 43 against the retractor spring 57. As a result, the retractor spring 57 provides a biasing force on the indenter 34 to hold the ferrule 18 within the aperture 21. The indenter 34 may be retracted for insertion of a ferrule 18 by the retracting collar 51, which may be attached to the indenter 34 by pin 53. Opposed longitudinally oriented slots 55 in a proximal portion of the nose extension 52 and opposed longitudinally oriented slots 61 in the distal portion of the pushrod 44 may be aligned to allow the pin 53 to pass through the slots 55,61 and through the indenter 34 to couple the indenter 34 to the retracting collar 51. Accordingly, the pin 53 is allowed to move longitudinally within the slots 55,61 to move the attached indenter 34 in concert with the retraction collar 51. The pushrod 43 may further include a neck-down region for capturing a retaining pin 47 inserted radially through the head 64, to reciprocally retain the pushrod 43 within the nose extension 52.

The plunger assembly 33 and the nosepiece assembly 62 are assembled into the nose 32 as described above. The proximal end of the nose 32 is inserted into the tool 10 so that the head 64 of the nose extension is entirely inserted within the body 28. The nose collar 72 is installed over the nose extension 52 to reciprocally retain the proximal end of the nose 32 within the body 28 at the flange 65. Accordingly, the nose 32 can be rotated about an elongate axis by depressing the nosepiece 22 in a direction to compress the return spring 56 to disengage the flange 65 from frictional contact with the nose collar 72, and allow the nose 32 to be rotatably positioned at a desired orientation.

To accommodate the special needs of a surgical environment, the tool may be made out of materials that are sterilizable with methods, such as autoclaving. For example, the tool may be formed from autoclavable stainless steel or aluminum components. To provide a tool that needs no lubricants that may compromise sterility, the machining tolerances may be increased over tolerances that are normally used in conventional tools. In addition, the tool may be assembled using flush mounted fasteners to ensure that the tool is snag resistant.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. A tool for surgically clamping a split sternum together comprising:
an elongated nosepiece extending from an end of the tool, a distal end of the nosepiece having an aperture, generally transverse to an elongate direction of the nosepiece, the aperture receiving a ferrule and allowing passage of a cable through the ferrule disposed in the aperture, the nosepiece further having a passageway extending from a proximal end of the nosepiece in the elongate direction to an intersection with the aperture; the proximal end of the nosepiece being retained in a body of the tool;
a plunger, comprising an indenter, a spring, and a pushrod member having a recess for receiving the spring and the a proximal portion of the indenter, the recess allowing reciprocating motion of the indenter therein, the plunger mounted in the passageway for reciprocating motion therein, so that in a first position, a distal portion of the indenter extends into the aperture sufficiently to hold a ferrule disposed therein;
a collar, attached to the indenter and slidingly disposed around the nosepiece, for allowing manual movement of the indenter to a second position away from the aperture to allow insertion of a ferrule in the aperture; and
an apparatus for advancing the plunger towards the aperture to compress a ferrule, disposed in the aperture, around a cable disposed through the ferrule.

2. The tool of claim 1, further comprising a wheel mounted for rotation about an axis of the body substantially perpendicular to an elongate axis of the body for retaining cable wrapped around the wheel and allowing tension to be applied to the cable by rotation thereof.

3. The tool of claim 1, wherein the pushrod member and the nose piece comprise alignable pairs of opposed longitudinal slots allowing longitudinal movement of a pin therethrough, the pin extending radially through the collar, the slots, and the indenter.

4. The tool of claim 1, wherein the apparatus for advancing the plunger comprises:
a toggle joint having a first arm for engaging the proximal end of the plunger; and
a lever for exerting a force on a second arm of the toggle joint to urge the first arm against the plunger.

5. The tool of claim 4, wherein the lever comprises a pivotable handle of a pliers-type hand tool.

6. A method of using the tool of claim 1, comprising:
providing a cable comprising a cable end fitting attached to a first end of the cable, the cable end fitting further comprising a cable passageway for receiving a second end of the cable therethrough;
forming a cable loop around the body parts by passing the second end of the cable around the body parts and through the cable end fitting;
disposing a ferrule in the aperture of the tool;
threading the second end of the cable through the ferrule;
positioning the ferrule against the cable end fitting;
tensioning the cable;
crimping the ferrule to the cable; and
cutting the cable flush with the ferrule.

7. A surgical tool for tensioning a cable positioned around a split sternum, for crimping a ferrule around the cable, and for terminating the cable, the tool including a tensioning actuator for gripping and tensioning the cable to a predetermined tension, and a crimping actuator for crimping the ferrule onto the cable when the predetermined tension has been reached, the crimping actuator being operative to sever a free end of the cable concurrently with crimping of the ferrule, the crimping actuator including an indenter to retain the ferrule within the crimping actuator during tensioning of the cable and crimping the ferrule.

8. A method of surgically clamping a split sternum together comprising:
looping a cable comprising a cable end fitting attached to a first end of the cable around the body parts;
passing a second end of the cable through the cable end fitting;
disposing a ferrule in a ferrule receiving aperture of a clamping tool;
threading the second end of the cable through the ferrule disposed in the aperture so that the ferrule abuts the cable end fitting;
wrapping the second end of the cable around a tensioning wheel of the clamping tool to apply a predetermined tension to the cable;
forcing an indenter of the clamping tool into the aperture against the ferrule to crimp the ferrule around the cable; and
forcing the ferrule past a shearing surface of the tool adjacent to an end of the ferrule away from the cable end fitting to cut the cable flush with the ferrule.

9. The method of claim 8, further comprising displacing the indenter from extending into the aperture to allow disposing the ferrule therein.

10. The method of claim 8, further comprising biasing the indenter against the ferrule after disposing the ferrule in the aperture.

11. The method of claim 8, further comprising passing the second end of the cable around the body parts more than one time before passing the second end through the cable end fitting.
